(19) United States
(12) Patent Application Publication
(10) Pub. No.: US 2026/0216030 A1
(43) Pub. Date: Jul. 30, 2026

(54) Compositions for Hormonally Impacted Skin

(71) Applicant: THE BOOTS COMPANY PLC, Nottingham (GB)

(72) Inventors: Michael David Bell, Alfreton Derbyshire (GB); Christopher Elms, Nottingham (GB); Victoria Newton, Beeston, Nottingham (GB)

(21) Appl. No.: 19/145,611

(22) PCT Filed: Jan. 3, 2024
(86) PCT No.: PCT/EP2024/025004

§ 371 Date: Jul. 3, 2025

(30) Foreign Application Priority Data
Jan. 5, 2023 (EP) 23020005.7

(51) Int. Cl. A61K 8/49; A61K 8/34; A61K 8/67; A61K 8/9783; A61Q 19/00

(52) U.S. Cl. CPC A61K 8/49; A61K 8/34; A61K 8/4926; A61K 8/675; A61K 8/9783; A61Q 19/00

(57) ABSTRACT

A composition comprising (i) a monocyclic sesquiterpene alcohol; (ii) vitamin B or a derivative thereof; and (iii) an amide of formula (I), (I), wherein X is CH or N, Y is CHR⁸ or O, n is 0, 1 or 2, preferably 1 or 2, R¹, R² and R³ are independently of each other selected from the group consisting of H, OH, a halogen atom, a carbamoyl group and C₁-C₆ alkyl group, and R⁴, R⁵, R⁶, R⁷ and R⁸ are independently of each other H or a C₁-C₆ alkyl group. The composition is suitable for topical application for treatment of hormonally impacted skin.

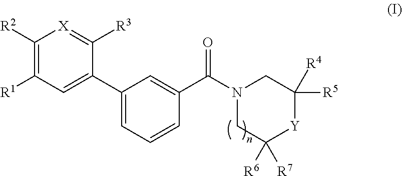

### FIELD OF THE INVENTION

The invention relates to the field of skin care. More particularly, the invention pertains to compositions, methods, and uses thereof in the treatment of hormonally impacted skin.

### BACKGROUND OF THE INVENTION

Cosmetically, healthy looking skin is characterised by skin that is well moisturised, smooth and blemish free, luminous and radiant, even in skin tone and non-oily. Throughout life, transient changes in for example lifestyle, mental well-being, sleep, nutrition, environmental exposure can lead to transient fluctuations in skin appearance driven primarily by an impact on epidermal homeostasis. Hormones represent an additional exposomal factor, whether produced naturally by endocrine glands coinciding with life stage or whether provided by supplementation as in contraception, performance enhancers and hormone replacement therapy (HRT) and there is growing evidence that hormones can impact on dermatologic mechanisms that will have an impact on cosmetic healthy skin concerns.

The most well researched hormonally-influenced or hormonally impacted skin condition is acne. This skin disease involves a complex cascade of events that are controlled by androgen hormones impacting on the pilosebaceous unit leading to increased sebum production and resulting inflammation. Fluctuating sex hormones are also implicated in pre-menstrual “acne” flares with peak testosterone levels associated with highest acne prevalence. However, the impact of hormone fluctuations is not limited to acne. Whilst sun exposure and genetics are no doubt important causal factors in hyperpigmentation, hormonal influences also play a significant role in its pathogenesis as seen by the increased prevalence with pregnancy, oral contraceptive use and other hormonal therapies. An increased susceptibility for melasma may also be linked to increases in local progesterone and oestrogen receptors in the skin.

### SUMMARY OF THE INVENTION

According to a first aspect of the invention, there is provided a composition comprising
(i) a monocyclic sesquiterpene alcohol;
(ii) vitamin B or a derivative thereof; and
(iii) an amide of formula (I)

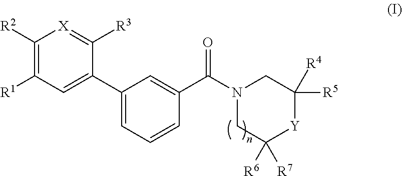

wherein X is CH or N,
Y is CHR⁸ or O, nis 0, 1 or 2, preferably 1 or 2,
R¹, R² and R³ are independently of each other selected from the group consisting of H, OH, a halogen atom, a carbamoyl group and C₁-C₆ alkyl group, and
R⁴, R⁵, R⁶, R⁷ and R⁸ are independently of each other H or a C₁-C₆ alkyl group.

The inventors have surprisingly found that the compositions (e.g. cosmetic compositions) defined herein have benefits for hormonally impacted skin e.g. skin conditions influenced by hormones or hormone fluctuations). The compositions are suitable for topical application to hormonally impacted skin. In particular, the compositions as defined herein have benefits in reducing the sensitivity of hormonally impacted skin e.g. how the skin reacts to hormone fluctuations. Skin sensitivity is a common condition in which skin is prone to itching and irritation, for example. This sensitivity can manifest, for example, as redness, dryness, increased sebum production, itchiness and/or blemishing.

According to a second aspect of the invention there is provided an in vitro method for assessing the effect of a test composition on hormonally impacted skin, the method comprising:
a) applying a hormonal insult to skin cells in vitro to yield hormonally impacted skin cells;
b) applying a test composition to the hormonally impacted skin cells; and
c) measuring a change in a property of the hormonally impacted cells.

The inventors have developed an in vitro method for assessing a target composition, which has benefits relative to in vivo methods, such as avoiding the need for human volunteers. Moreover the method allows test compositions to be tested in a fair and consistent manner over a long period of time. Measuring a change in the property of the hormonally impacted cells may comprise monitoring a biomarker. Biomarkers of cell health and performance include but are not limited to proliferation rate; oxidative stress (malondialdehyde (MDA) is a marker for oxidative stress; inflammatory biomarkers (e.g. inflammatory cytokines); and cell viability.

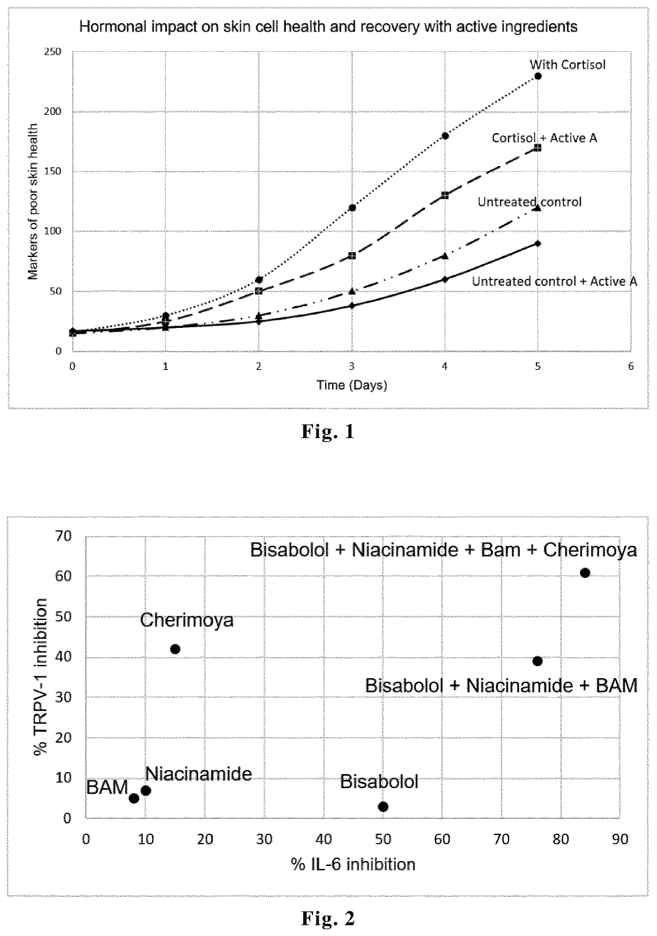

FIG. 1 is a schematic hypothetical diagram to illustrate the method of the second aspect. A hormonal insult (e.g. cortisol) is applied to skin cells in vitro to yield hormonally impacted cells. A test composition (e.g. Active A) is applied to the hormonally impacted skin cells and a property is measured (e.g. a marker of poor skin health). In this example, the test composition (Active A) reduces the adverse effects of the hormone (cortisol), as shown by the “With Cortisol” line being higher than the “Cortisol+Active A” line.

The compositions of the present invention may be employed for purely cosmetic purposes, as defined in the third aspect of the invention. Alternatively, the composition may be for use as a medicament, e.g. for a person with a medical condition (e.g. eczema, psoriasis, dermatitis, rosacea or acne), as defined in the fourth to sixth aspects of the invention.

According to a third aspect of the invention there is provided a cosmetic method comprising topically administering the composition of the first aspect. The cosmetic method is for preventing and/or treating skin sensitivity.

According to a fourth aspect of the invention there is provided the composition of the first aspect for use as a medicament.

According to a fifth aspect of the invention, there is provided a kit comprising the composition of the first aspect together with a pharmaceutical composition.

According to a sixth aspect of the invention, there is provided the kit of the fifth aspect for use as a medicament. In particular for treating a skin condition e.g. eczema, psoriasis, dermatitis, rosacea or acne.

It will be appreciated that the composition of the first aspect may be employed before the pharmaceutical composition; simultaneously with the pharmaceutical composition; or after the pharmaceutical composition.

According to a seventh aspect of the invention there is provided a method for treating a skin condition comprising
(i) topically administering the composition of the first aspect (to the skin of a person in need thereof); and
(ii) administering a pharmaceutical composition.

It will be appreciated that the composition of the first aspect may be employed before the pharmaceutical composition; simultaneously with the pharmaceutical composition; or after the pharmaceutical composition. The pharmaceutical composition may be applied topically to the same area of skin as the first composition. Alternatively, the pharmaceutical composition may be orally administered (e.g. a tetracyclines).

### DETAILED DESCRIPTION OF THE INVENTION

Unless otherwise indicated, a reference to the composition of the invention is a reference to the compositions of all aspects of the invention.

### A Monocyclic Sesquiterpene Alcohol

Sesquiterpenes are a class of terpenes that consist of three isoprene units and often have the molecular formula C₁₅H₂₄. Sesquiterpenes may be acyclic or contain rings. The present invention employs an alcohol derivative of a monocyclic (single ring) sesquiterpene. For example, the monocyclic sesquiterpene alcohol may be α-bisabolol (shown below, left) or β-bisabolol (shown below, right). Synthetic bisabolol is usually a racemic mixture: α-(±)-bisabolol

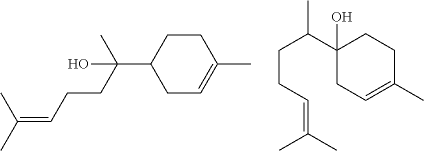

The monocyclic sesquiterpene alcohol (e.g. α-bisabolol or β-bisabolol) may be present in the composition in an amount by weight of at least 0.001%, at least 0.005%, at least 0.01%, at least 0.05%, at least 0.1%, at least 0.5% or at least 1%; and/or the monocyclic sesquiterpene alcohol (e.g. α-bisabolol or β-bisabolol) may be present in the composition in an amount by weight of 2% or less, 1% or less, 0.5% or less, 0.1% or less, 0.05% or less or 0.01% or less.

In particular, the monocyclic sesquiterpene alcohol (e.g. α-bisabolol or β-bisabolol) may be present in the composition in an amount by weight of from 0.001% to 2%, from 0.01% to 1%, or from 0.05% to 0.5%.

### Vitamin B or Vitamin B Derivative

The composition comprises vitamin B (e.g. vitamin B1 to vitamin B12) or a vitamin B derivative (e.g. a derivative of vitamin B1 to B12, such as a vitamin B3). In particular, the composition may comprise a vitamin B3 compound, such as niacinamide.

Vitamin B3 and niacin, are the common names for nicotinic acid. The physiologically active form of niacin is niacinamide. Niacin and niacinamide (nicotinamide or nicotinic acid amide) function in the body as a component of two coenzymes: nicotinamide adenine dinucleotide (NAD) and nicotinamide adenine dinucleotide phosphate (NADP).

The compositions used in the present invention may comprise a safe and effective amount of a (natural or synthetic) vitamin B3 compound. As used herein, “vitamin B3 compound” means a compound having the formula:

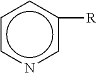

wherein R is —CONH₂ (i.e., niacinamide), —COOH (i.e., nicotinic acid) or —CH₂OH (i.e., nicotinyl alcohol); derivatives thereof; and salts of any of the foregoing. Exemplary derivatives of the foregoing vitamin B3 compounds include nicotinic acid esters, including non-vasodilating esters of nicotinic acid, nicotinyl amino acids, nicotinyl alcohol esters of carboxylic acids, nicotinic acid N-oxide and niacinamide N-oxide.

Suitable esters of nicotinic acid include nicotinic acid esters of C1-C22, preferably C1-C16, more preferably C1-C6 alcohols. The alcohol may be straight-chain or branched chain, cyclic or acyclic, saturated or unsaturated (including aromatic), and substituted or unsubstituted. Preferred esters are those which do not yield a visible flushing response after application to the skin. Alternatively, a nicotinic acid material which does produce a flushing response can be used, if used at a lower dose to reduce the flushing effect. Non-flushing esters of nicotinic acid include tocopherol nicotinate and inositol hexanicotinate. Tocopherol nicotinate is preferred.

Other derivatives of the vitamin B3 compound are derivatives of niacinamide resulting from substitution of one or more of the amide group hydrogens. Non-limiting examples of derivatives of niacinamide useful herein include nicotinyl amino acids, derived, for example, from the reaction of an activated nicotinic acid compound (e.g., nicotinic acid azide or nicotinyl chloride) with an amino acid, and nicotinyl alcohol esters of organic carboxylic acids (e.g., C1-C18). Specific examples of such derivatives include nicotinuric acid and nicotinyl hydroxamic acid.

Exemplary nicotinyl alcohol esters include nicotinyl alcohol esters of carboxylic acids salicylic acid, acetic acid, glycolic acid, palmitic acid and the like. Other suitable vitamin B3 compounds are selected from the group consisting of 2-chloronicotinamide, 6-aminonicotinamide, 6-methylnicotinamide, n-methyl-nicotinamide, n,n-diethylnicotinamide, n-(hydroxymethyl)-nicotinamide, quinolinic acid imide, nicotinanilide, n-benzylnicotinamide, n-ethylnicotinamide, nifenazone, nicotinaldehyde, isonicotinic acid, methyl isonicotinic acid, thionicotinamide, nialamide, 1-(3-pyridylmethyl) urea, 2-mercaptonicotinic acid, nicomol, niaprazine and mixtures thereof.

Examples of the above vitamin B3 compounds are well known in the art and are commercially available from a number of sources, e.g., the Sigma Chemical Company (St. Louis, MO); ICN Biomedicals, Inc. (Irvin, CA) and Aldrich Chemical Company (Milwaukee, WI).

The compositions of the present invention preferably comprise one or more vitamin B3 compounds. Preferred vitamin B3 compounds are niacinamide and tocopherol nicotinate. Niacinamide is more preferred.

Salts of the vitamin B3 compound are also useful herein. Non-limiting examples of salts of the vitamin B3 compound useful herein include organic or inorganic salts, such as inorganic salts with anionic inorganic species (e.g., chloride, bromide, iodide, carbonate, preferably chloride), and organic carboxylic acid salts (including mono-, di- and tri-C1-C18 carboxylic acid salts, e.g., acetate, salicylate, glycolate, lactate, malate, citrate, preferably monocarboxylic acid salts such as acetate).

The vitamin B3 compound may be included as the substantially pure material, or as an extract obtained by suitable physical and/or chemical isolation from natural sources. The vitamin B3 compound is preferably substantially pure, more preferably essentially pure.

The vitamin B or vitamin B derivative (e.g. vitamin B3 compound, such as niacinamide) may be present in the composition in an amount by weight of at least 0.01%, at least 0.05%, at least 0, 1%, at least 0.5%, at least 1%, at least 2%, at least 3%, at least 4% or at least 5%; and/or the vitamin B or vitamin B derivative (e.g. vitamin B3 compound such as niacinamide) may be present in the composition in an amount by weight of 5% or less, 4% or less, 3% or less, 2% or less, 1% or less, 0.5% or less, 0.1% or less, or 0.05% or less. Where more than one vitamin B/vitamin B derivative is present, the values apply to the total amount of vitamin B or vitamin B derivative.

In particular, the vitamin B or vitamin B derivative (e.g. niacinamide) may be present in the composition in an amount by weight of from 0.01 to 5%, 0.05% to 3%, from 0.1% to 3%, 0.1% to 2%, from 0.5% to 1%, or from 0.5% to 2%.

### Amide of Formula (I)

The composition comprises an amide of formula (I)

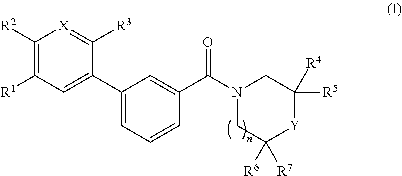

wherein X is CH or N,
Y is CHR⁸ or O, nis 0, 1 or 2, preferably 1 or 2,
R¹, R² and R³ are independently of each other selected from the group consisting of H, OH, a halogen atom, a carbamoyl group and C₁-C₆ alkyl group, and
R⁴, R⁵, R⁶, R⁷ and R⁸ are independently of each other H or a C₁-C₆ alkyl group.

The amide of formula (I) may contain only one residue selected from the group consisting of OH, a halogen atom and a carbamoyl group.

The C₁-C₆ alkyl group may be an unbranched C₁-C₃ alkyl group, more preferably a C₁-C₂ alkyl group, most preferably a methyl group.

The halogen atom may be F or Cl.

The amide of formula (I) may be selected from (4-methylpiperidin-1-yl) (3-(6-methylpyridin-3-yl)phenyl) methanone, (4′-hydroxy-[1,1′-biphenyl]-3-yl) (4-methylpiperidin-1-yl) methanone, (4′-fluoro-[1,1-biphenyl]-3-yl) (4-methylpiperidin-1-yl) methanone, (3′-fluoro-4′-methyl-[1,1-biphenyl]-3-yl) (4-methylpiperidin-1-yl) methanone, (2′-chloro-[1,1′-biphenyl]-3-yl) (4-methylpiperidin-1-yl) methanone, (4′-methyl-[1,1′-biphenyl]-3-yl) (4-methylpiperidin-1-yl) methanone, (4′-chloro-[1,1′-biphenyl]-3-yl) (4-methylpiperidin-1-yl) methanone, (3,3-dimethylpiperidin-1-yl) (3′-fluoro-4′-methyl-[1,1′-biphenyl]-3-yl) methanone, 3′-(4-methylpiperidine-1-carbonyl)-[1,1′-biphenyl]-4-carboxamide, (3′-hydroxy-[1,1′-biphenyl]-3-yl) (4-methylpiperidin-1-yl) methanone, 3′-(4-methylpiperidine-1-carbonyl)-[1,1-biphenyl]-3-carboxamide, (2,2-dimethylmorpholino) (3′-fluoro-4′-methyl-[1,1-biphenyl]-3-yl) methanone, (2,6-dimethylmorpholino) (3′-fluoro-4′-methyl-[1,1-biphenyl]-3-yl) methanone, (2,6-dimethylmorpholino) (4′-methyl-[1,1 biphenyl]-3-yl) methanone, azepan-1-yl (3′-fluoro-4′-methyl-[1,1′-biphenyl]-3-yl) methanone, azepan-1-yl (4′-chloro-[1,1′-biphenyl]-3-yl) methanone, azepan-1-yl (4′-methyl-[1,1′-biphenyl]-3-yl) methanone, azepan-1-yl (4′-hydroxy-[1,1′-biphenyl]-3-yl) methanone, azepan-1-yl (3-(6-methylpyridin-3-yl)phenyl) methanone, [1,1′-biphenyl]-3-yl (azepan-1-yl) methanone, or azepan-1-yl (3′,4′-dimethyl-[1,1′-biphenyl]-3-yl) methanone.

The amide of formula (I) may be [1,1′-biphenyl]-3-yl (azepan-1-yl) methanone, also known as [1,1′-biphenyl]-3-yl (hexahydro-1H-azepin-1-yl)-methanone (INCI name: Biphenyl Azepanyl Methanone, CAS No: 1910069-14-5, also known as BEL-EVEN®).

The amide of formula (I) (e.g. biphenyl azepanyl methanone) may be present in the composition in an amount by weight of at least 0.0005%, at least 0.001%, at least 0.005%, at least 0.01% or at least 0.05%; and/or the amide of formula (I) (e.g. biphenyl azepanyl methanone) may be present in the composition in an amount by weight of 1% or less, 0.5% or less, 0.1% or less, 0.05% or less, 0.01% or less, or 0.05% or less.

In particular, the amide of formula (I) (e.g. biphenyl azepanyl methanone) may be present in the composition in an amount by weight of from 0.001% to 1%, from 0.01% to 0.5%, or from 0.05% to 0.1%.

Methods for the preparation of amides of formula (I) are disclosed in WO2017012890. WO2021089501 describes a method of preventing and/or treating dry skin and loss of natural oiliness in a person in need thereof by topically administering an amide of formula (I). WO2021089501 teaches the use of an amide of formula (I) to stimulate the sebum production in sebaceous glands.

### Annona Extract

The composition may additionally comprise an Annona extract.

Annona is a genus of flowering plants in the pawpaw/sugar apple family, Annonaceae. The Annona extract may be a cherimoya extract, a soursop extract or a sugar-apple extract.

The Annona extract may be a fruit extract or a flower extract. In some circumstances the Annona extract may be a seed or bark extract.

Preferably, the composition comprises an Annona fruit extract, i.e. extracted from the fruit (not the seed or bark, which may be poisonous) of a plant of the Annona genus.

The cherimoya (Annona cherimola) is a species of edible fruit-bearing plant in the genus Annona, and commonly described as a custard apple. The composition may comprise an Annona cherimola fruit extract or flower extract. Preferably the composition comprises an Annona cherimola fruit extract.

Soursop is the fruit of Annona muricata. The composition may comprise an Annona muricata fruit extract or flower extract

The sugar-apple or sweet-sop is the fruit of Annona squamosa. The composition may comprise an Annona squamosa fruit extract or flower extract

The Annona extract (e.g. Annona cherimola fruit extract) may be present in the composition in an amount by weight of at least 0.01%, at least 0.05%, at least 0.1%, at least 0.5% or at least 1%; and/or the Annona extract (e.g. Annona cherimola fruit extract) may be present in the composition in an amount by weight of 6% or less, 4% or less, 2% or less, 1% or less, 0.5% or less, or 0.1% or less.

In particular, the Annona extract (e.g. the Annona cherimola fruit extract) may be present in the composition in an amount by weight of from 0.1% to 5%, from 0.5% to 4%, or from 1% to 3%.

### Ginseng Extract

The composition may additionally comprise a ginseng extract.

Ginseng plants belong to the genus Panax. The composition of the invention may comprise Panax ginseng (Korean ginseng), Panax notoginseng (South China ginseng), or Panax quinquefolius (American ginseng). The composition preferably comprises Panax ginseng.

The ginseng extract may be a root extract, such as Panax ginseng root extract.

The ginseng extract (e.g. Panax ginseng root extract) may be present in the composition in an amount by weight of at least at least 0.005%, at least 0.01%, at least 0.02% or at least 0.03%; and/or the ginseng extract (e.g. Panax ginseng root extract) may be present in the composition in an amount by weight of 1% or less, 0.5% or less, 0.1% or less, 0.08% or less, or 0.5% or less.

In particular, the ginseng extract (e.g. Panax ginseng root extract) may be present in the composition in an amount by weight of from 0.01% to 1%, from 0.02% to 0.5%, or from 0.03% to 0.1%.

### Trifolium Extract

The composition may comprise a Trifolium extract.

Trifolium is a plant genus, which is commonly known as clover. The trifolium extract may be an extract of Trifolium pratense (red clover) or Trifolium repens (white clover), for example. The trifolium extract may be a flower or leaf extract, for example.

The trifolium extract may be an extract from a flower of the genus Trifolium, such as a Trifolium pratense (red clover) flower extract. The trifolium extract may be an extract from a leaf of the genus Trifolium, such as a Trifolium pratense (red clover) leaf extract.

The Trifolium extract (e.g. red clover flower extract) may be present in the composition in an amount by weight of at least at least 0.05%, at least 0.1%, at least 0.15% or at least 2%; and/or the Trifolium extract (e.g. red clover flower extract) may be present in the composition in an amount by weight of 3% or less, 2% or less, 1% or less, 0.8% or less, or 0.5% or less.

In particular, the Trifolium extract (e.g. red clover flower extract) may be present in the composition in an amount by weight of from 0.05% to 2%, from 0.1% to 1%, or from 0.2% to 0.5%.

### Ceramide

Ceramides are a family of waxy lipid molecules. A ceramide is composed of sphingosine and a fatty acid.

The composition may comprise at least one ceramide. The at least one ceramide may comprise ceramide 1 (ceramide EOP), ceramide 2 (ceramide NG), ceramide 3 (ceramide NP) and/or ceramide 6 (ceramide AP). The composition preferably comprises ceramide 3 (ceramide NP), also known as N-oloeoyl-4-hydroxysphinganine. The composition preferably comprises ceramide 6 (ceramide AP).

The composition may comprise at least two ceramides, such as ceramide 3 and ceramide 6. The composition may comprise at least 3 ceramides, such as ceramide 1, ceramide 3, and ceramide 6.

The at least one ceramide (e.g. combined amounts of ceramide 1, 3 and 6) may be present in the composition in an amount by weight of at least at least 0.003%, at least 0.005%, at least 0.008% or at least 0.01%; and/or the at least one ceramide (e.g. combined amounts of ceramides 1, 3 and 6) may be present in the composition in an amount by weight of 0.1% or less, 0.05% or less, 0.03% or less, or 0.01% or less.

In particular, the at least one ceramide (e.g. combined amounts of ceramides 1, 3 and 6) may be present in the composition in an amount by weight of from 0.003% to 0.03% or from 0.005% to 0.02%.

### Saccharide Isomerate

The composition may comprise saccharide isomerate. Saccharide isomerate consists mainly of glucose, fructose mannose and galactose.

Saccharide isomerate mimics NMF (the Natural Moisturising Factor) found in human skin and is derived from plant based glucose. Saccharide isomerate is commercially available under the trademark name PENTAVITIN® from DSM Nutritional Products Ltd.

The saccharide isomerate may be present in the composition in an amount by weight of at least at least 0.05%, at least 0.1%, at least 0.2% or at least 0.3%; and/or the saccharide isomerate may be present in the composition in an amount by weight of 2% or less, 1% or less, 0.8% or less, 0.6% or less or 0.4% or less.

In particular, the saccharide isomerate may be present in the composition in an amount by weight of from 0, 1% to 0.9% or from 0.2% to 0.6%.

### Hyaluronic Acid or Salt Thereof

The composition may comprise hyaluronic acid or a salt thereof, such as sodium hyaluronate or potassium hyaluronate. The composition preferably comprises sodium hyaluronate.

The hyaluronic acid or salt thereof (e.g. sodium hyaluronate) may be present in the composition in an amount by weight of at least at least 0.01%, at least 0.02%, at least 0.05% or at least 0.1%; and/or the hyaluronic acid or salt thereof (e.g. sodium hyaluronate) may be present in the composition in an amount by weight of 1% or less, 0.5% or less, 0.2% or less, or 0.1% or less.

In particular, the hyaluronic acid or salt thereof (e.g. sodium hyaluronate) may be present in the composition in an amount by weight of from 0.01% to 0.2% or from 0.03% to 0.1%.

### Other Components

The compositions of the invention may be aqueous or non-aqueous and comprise of a single-phase system or multiple phase system. The (cosmetic) compositions may include but is not limited to liquids, gels, balms, oils or solids. Single or multiple phase compositions are envisaged. Multiple phase systems include but are not limited to microemulsions, emulsions, and products with discrete separate phases. Emulsions include water-in-oil, oil-in-water emulsions and multiple emulsions (water in oil in water or oil in water in oil for example). Products with discrete separate phases include bi or triphasic systems where the individual water or oil phases can be visibly seen.

Where the (cosmetic) composition is aqueous, it preferably comprises from 20% to 95% by weight water. In a preferred embodiment, aqueous compositions comprise from 40% to 90% by weight water. In a preferred embodiment, aqueous compositions comprise from 60% to 85% by weight water. Where the composition is non-aqueous it preferably comprises 0% to up to 10% water, more particularly from 0.1 to 8%, most preferably from 0.5 to 5% water.

Where the cosmetic composition is an emulsion, it comprises an oil and a water phase. The oil phase of an emulsion can be provided by any suitable oily component. Suitable oils for the oil phase may comprise for example: a) hydrocarbon oils, such as paraffin or mineral oils; b) waxes, such as beeswax or paraffin wax; c) natural oils, such as sunflower oil, apricot kernel oil, shea butter or jojoba oil; d) silicone oils, such as dimethicone, silicone elastomer, cyclomethicone or cetylidimethicone; e) fatty acid esters and ethers, such as isopropyl palmitate or isopropyl myristate and polypropylene glycol-15 stearyl ether; f) fatty alcohols, such as cetyl alcohol or stearyl alcohol; or g) mixtures thereof, for example, the blend of waxes available commercially under the trade name Cutina (BASF).

The emulsion may comprise 0.1% to 55% by weight of the emulsion of oil phase. In one embodiment, the emulsion may comprise 3% to 25% by weight of the emulsion of oil phase, more preferably from 5% to 20% by weight of the emulsion of oil phase. In an alternative embodiment, the emulsion may comprise 10% to 50% by weight of the emulsion of oil phase, more preferably from 25-50% by weight of the emulsion of oil phase.

The compositions of the invention may comprise an emulsifier. Suitable emulsifiers include all those suitable for the purpose and known by those skilled in the art for use in skin care products. Preferably these emulsifiers have an HLB value of or less than 14, more preferably from 2 to 14, and still more preferably from 4 to 14.

In some embodiments, the composition further comprises one or more antioxidant agents selected from a) ascorbic acid its salts, esters, glucosides and glucosamines, particularly sodium ascorbyl phosphate, magnesium ascorbyl phosphate, ascorbyl palmitate and ethyl ascorbic acid b) vitamin E (tocopherol) and its esters, particularly tocopheryl acetate, as well as Dimethyl methoxy chromanol which is a synthetic analogue of gamma tocopherol, available from Lipotec S.A. polygon Industrial Camri Ral, under the tradename Lipochroman-6 c) herbal extracts (not including Annona, ginseng and Trifolium extracts), particularly gingko biloba, such as that available under the trade name “Gingko Biloba Leaf Powder” from Univar PLC, morus alba, such as that available under the trade name “Mulberry Concentrate” from Solabia, Origanum vulgare, such as that available under the trade name “Pronalen Origanum HSC” from S Black Ltd, birch extract such as those available from Cosmetochem (U. K.) Ltd under the trade names “Super Herbasol Extract Birch” and “HP Herbasol Betula” and those available from Blagden Chemicals under the tradenames “Phytelene of Birch” and “Aqueous Spray Dried Birch”, Camellia sinensis, such as that available under the trade name “Herbal Extract Green Tea 75% Solids” from Nichimen Europe, Rosmarinus officinalis, such as that available under the trade name “Pronalen Rosemary” from S. Black, Acerola cherry powder, such as that available as Acerola PE from Gee Lawson, Emblica extract sold under the tradename Emblica™ by Merck Speciality chemicals, and Grape Seed oil, such as that available from Chesham Chemicals Limited.

The amounts of the antioxidant agents used in the cosmetic compositions are expressed as dry weights, as understood by a man skilled in the art. The total amount of antioxidant agents optionally present in the cosmetic compositions may range from 0.005% to 10% by weight, preferably 0.01% to 5%, most preferably 0.05% to 1.0% by weight of the total weight of the cosmetic composition.

The compositions of the invention may optionally comprise a skin conditioning agent. Said skin conditioning agents may preferably be selected from the group consisting of humectants, emollients, moisturisers, or mixtures thereof. Where present, they are preferably present at a level of from 0.01% to 20%, more preferably from 0.1% to 10%, most preferably from 0.5% to 7% by weight of the cosmetic composition.

Preferred skin conditioning agents are selected from the group consisting of guanidine, urea, glycolic acid and glycolate salts, salicylic acid, lactic acid and lactate salts, aloe vera, shea butter, polyhydroxy alcohols, such as sorbitol, mannitol, xylitol, erythritol, glycerol, hexanetriol, butanitriol, (di) propylene glycol, butylene glycol, hexylene glycol, polyethylene glycol, sugars (e.g. fructose, glucose, xylose, honey, mannose, xylose), gluconodeltalactone, and starches and their derivatives, pyrrolidone, carboxylic acid, lactamide monoethanolamine, acetamide monoethanolamine, panthenol, allantoin and mixtures thereof.

The composition may comprise a humectant that is a diol or a triol. The composition may comprise a diol selected from the group consisting of pentylene glycol, caprylyl glycol, butylene glycol, di-propylene glycol, af ethylhexylglycerine, propanediol, hexenediol, glycerol, butylene glycol, propylene glycol, isoprene glycol, dipropylene glycol, pentylene glycol, hexylene glycol, polypropylene glycol, butylene glycol, polyethylene glycol, sorbitol, glucitol, mannitol, hydroxypropyl sorbitol, erythritol, threitol, pentaerythritol, xylitol. The composition may comprise a triol selected from the group consisting of hexanetriol, glycerine, ethoxylated glycerin, propoxylated glycerin and mixtures thereof.

The composition may comprise propylene glycol, also known as propanediol (e.g. 0.3 to 2 wt % propylene glycol); butylene glycol (e.g. 0.1 to 0.5 butylene glycol); glycerin (e.g. 3 to 10% glycerin); panthenol (e.g. 0.1 to 0.9 wt % panthenol); and/or caprylyl glycol (e.g. 0.1 to 0.9%);

The compositions of the invention may comprise one or more vitamins, in addition to the Vitamin B/Vitamin B derivative. The compositions may comprise ascorbates, for example vitamin C, vitamin C derivatives, ascorbic acid, ascorbyl glucoside, ascorbyl palmitate, magnesium ascorbyl phosphate, sodium ascorbyl phosphate and ethyl ascorbic acid.

In some embodiments the cosmetic compositions may comprise vitamin K, vitamin K derivatives, vitamin H, vitamin D, vitamin D derivatives and mixtures thereof. In an alternative embodiment of the present the cosmetic compositions comprise vitamin E, vitamin E derivatives such as tocopherol and tocopheryl acetate, and provitamins thereof, such as panthenol and mixtures thereof.

In a further embodiment, the cosmetic compositions of the invention comprise retinoid compounds, including retinoic acid, retinaldehyde, retinol and derivatives thereof. In one embodiment the cosmetic compositions comprise retinyl palmitate, retinyl acetate, retinyl retinoate, retinyl proprionate, retinyl ascorbate, retinyl linoleate, retinyl retinoate, retinyl sunflowerseedate and mixtures thereof.

The vitamin compounds may be included as the substantially pure material, or as an extract obtained by suitable physical and/or chemical isolation from natural (e.g. plant) sources. In one embodiment, when vitamin compounds are present in the cosmetic compositions of the instant invention, the cosmetic compositions comprise from about 0.0001% to 50%, more preferably from 0.001% to 10%, still more preferably from 0.01% to 8%, and still more preferably from 0.1% to 5%, by weight of the cosmetic composition, of the vitamin compound.

The compositions of the invention may optionally comprise a sunscreen component. The sunscreen may comprise organic or inorganic sun filters or a combination of the two. Suitable inorganic sun filters include those selected from the group consisting of microfine titanium dioxide, microfine zinc oxide, boron nitride and mixtures thereof. Suitable organic sunscreens include those selected from the group consisting of: a) p-aminobenzoic acids, their esters and derivatives (for example, 2ethylhexyl p-dimethylaminobenzoate), b) methoxycinnamate esters (for example, 2-ethylhexyl p-methoxycinnamate, 2-ethoxyethyl p-methoxycinnamate or a, p-di-(p-methoxycinnamoyl)-a′-(2ethylhexanoyl)-glycerin, c) benzophenones (for example oxybenzone), d) dibenzoylmethanes such as 4-(tert-butyl)-4′-methoxydibenzoylmethane, e) 2-phenylbenzimidazole-5 sulfonic acid and its salts, f) alkyl-ss, ss-diphenylacrylates for example alkyl a-cyano-ss, ss-diphenylacrylates such as octocrylene, g) triazines such as 2,4,6-trianilino-(p-carbo-2-ethyl-hexyl-1-oxi)-1,3,5-triazine, h) camphor derivatives such as methylbenzylidene camphor and i) mixtures thereof. Other preferred sunscreen ingredients include those selected from the group consisting of homosalate, Ethylhexyl salicylate, Diethylhexylbutamido triazone, Bis-ethylhexyloxyphenol methoxyphenyl triazine, Diethylamino hydroxybenzoyl hexyl benzoate, Butyl methoxydibenzoylmethane, Methylene bis-benzotriazoyl tetramethylbutylphenol, Polysilicone-15 and mixtures thereof. A sunscreen agent is optionally present in an amount from 0.1 to 10% by weight of the cosmetic composition.

The compositions of the invention may also optionally comprise one or more of the following optional ingredients. The cosmetic compositions of the invention may comprise preservatives such as 2-bromo2-nitropropane-1,3-diol (bronopol, which is available commercially under the trade name Myacide®), benzyl alcohol, diazolidinyl urea, imidazolidinyl urea, methyl paraben, phenoxy ethanol, ethyl paraben, propyl paraben, sodium methyl paraben, sodium dehydroacetate, polyhexamethylenebiguanide hydrochloride, sodium benzoate, chlorhexidine digluconate, isothiazolone and sodium propyl paraben, suitably in an amount of from 0.01% to 10% by weight of the cosmetic composition.

The compositions of the invention may comprise thickeners, viscosity modifying agents and/or gelling agents, such as acrylic acid polymers e.g. available commercially under the trade name Carbopol, Ultrez or Novethix (Lubrizol) or Sepigel, Sepiplus and Simulgel (Seppic). Modified celluloses may also be added, for example hydroxyethylcellulose available commercially under the trade name Natrosol (Hercules) or hydroxypropylmethyl cellulose, amine oxides, block polymers of ethylene oxide and propylene oxide (for example, those available from BASF Wyandotte under the trade name “Pluronic”®), PVM, MA, or a decadiene crosspolymer (available under the trade name Stabilez 60), ethoxylated fatty alcohols, salt (magnesium chloride, sodium chloride), Aristoflex AVC (Clariant), phthalic acid amide, xanthan gum, sodium polyacrylate, polyvinyl alcohols, fatty alcohols and alkyl galactomannans available under the trade name N-Hance from Hercules, suitably in an amount of from 0.1% to 10% by weight of the cosmetic composition.

Sequestering agents may be added to the cosmetic compositions, such as ethylenediamine tetraacetic acid and salts thereof, suitably in an amount of from 0.005% to 0.5% by weight of the cosmetic composition.

The compositions may also include waxes such as cocoa butter or shea butter suitably in an amount of from 1% to 99% (e.g. 3 to 10%) by weight of the cosmetic composition

The cosmetic compositions may also comprise suitable, cosmetically acceptable diluents, carriers and/or propellants such as dimethyl ether.

The compositions may also include pearlising agents such as stearic monoethanolamide and/or mica, suitably in an amount of from 0.01% to 10% by weight of the cosmetic composition.

The compositions may include perfumes. In some embodiments the perfumes are present in an amount of from 0.01% to 2% by weight of the cosmetic compositions, as may water soluble dyes such as tartrazine, suitably in an amount of from a trace amount (such as 1×10-5%) to 0.1% by weight of the cosmetic composition.

The compositions may also include pH adjusting agents such as sodium hydroxide, aminomethyl propanol, triethanolamine, suitably in an amount of from 0.01% to 10% by weight of the cosmetic composition. The cosmetic compositions may be buffered by means well known in the art, for example by use of buffer systems comprising succinic acid, citric acid, lactic acid, and acceptable salts thereof, phosphoric acid, mono- or disodium phosphate and sodium carbonate. Suitably, the cosmetic composition may have a pH between 3 and 10, preferably between 4 and 8, more preferably from 4.5 to 6.

### Example Formulations

The compositions of the invention comprise (i) a monocyclic sesquiterpene alcohol; (ii) vitamin B or a derivative thereof; and (iii) an amide of formula (I). The composition may additionally comprise (iv) an Annona fruit extract.

Component (i)—the monocyclic sesquiterpene alcohol (e.g. α-bisabolol or β-bisabolol)—may be present in the composition in an amount by weight that is less than component (ii)—the vitamin B or a derivative thereof (e.g. niacinamide). In particular, component (i) may be present in the composition in an amount by weight that is 3 to 30% of the amount of component (ii).

Component (iii)—the amide of formula (I) (e.g. biphenyl azepanyl methanone) may be present in the composition in an amount by weight that is less than component (i)—the monocyclic sesquiterpene alcohol (e.g. α-bisabolol or β-bisabolol). In particular, component (iii) may be present in the composition in an amount by weight that is 3 to 30% of the amount of component (i).

Component (iii)—the amide of formula (I) (e.g. biphenyl azepanyl methanone) may be present in the composition in an amount by weight that is less than component (ii)—the vitamin B or a derivative thereof (e.g. niacinamide). In particular, component (iii) may be present in the composition in an amount by weight that is 0.3 to 3% of the amount of component (ii).

When present, component (iv)—the Annona fruit extract (e.g. Annona cherimola fruit extract) may be present in the composition in an amount by weight that is greater than each of components (i), (ii) and (iii). In particular, component (i) may be present in the composition in an amount by weight that is 3 to 30% of the amount of component (iv); component (ii) may be present in the composition in an amount by weight that is 30 to 99% of the amount of component (iv); and/or component (iii) may be present in the composition in an amount by weight that is 0.3 to 3% of the amount of component (iv).

The composition may comprise bisabolol, niacinamide and biphenyl azepanyl methanone. More preferably the composition comprises bisabolol, niacinamide, biphenyl azepanyl methanone and Annona cherimola fruit extract. Specific examples of the amounts by weight (wt %) are set out in the table below.

| | Example A | Example B | Example C |
|---|---|---|---|
| Bisabolol | 0.03 to 0.3 | 0.05 to 0.15 | 0.01 to 0.1 |
| Niacinamide | 0.3 to 3.0 | 0.5 to 1.5 | 0.1 to 1.0 |
| Biphenyl azepanyl | 0.01 to 0.2 | 0.005 to 0.015 | 0.001 to 0.01 |
| methanone | | | |
| Annona cherimola | 2.0 to 5.0 | 2.5 to 3.5 | 1.0 to 2.0 |
| fruit extract | | | |

### Hormonally Impacted Skin Cells

The method of the second aspect may be employed to assess the effect of a test composition on hormonally impacted skin cells.

The skin cells may be keratinocytes. Most of the cells (90-95%) in the epidermis are keratinocytes.

The skin cells may be cultured in a cell line, such as the HaCaT cell line. HaCaT is a spontaneously transformed aneuploid immortal keratinocyte cell line from adult human skin.

The skin cells may be cultured in a primary cell culture. Primary cell culture is the ex vivo culture of cells freshly obtained from a multicellular organism, as opposed to the culture of immortalized cell lines.

Applying a hormonal insult may comprise applying a hormone at a fixed concentration or applying a hormone at a concentration that varies over time. For example, applying a hormonal insult may comprise applying a hormone and subsequently reducing or withdrawing the hormone. This can be useful to simulate hormone fluctuations.

Applying a hormonal insult may comprise applying a steroid hormone or a peptide hormone. Applying a hormonal insult may comprise applying a human hormone. The hormone may be a glucocorticoid (e.g. cortisol, dexamethasone), a mineralocorticoid, an androgen (e.g. testosterone), an oestrogen (e.g. oestradiol), a progestogen (e.g. progesterone). The hormone may be a peptide hormone (e.g. melanocyte stimulating hormone (MSH), glucagon, insulin, insulin-like growth factor 1 (IGF-1), or a glycoprotein hormone (e.g. a Gonadotropin such as Luteinising hormone (LH) or Follicle stimulating hormone (FSH)) or a thyroid hormone, such as thyroxine.

### Oestrogen

Applying a hormonal insult may comprise applying or withholding oestrogen. The menopause represents a transition period when oestrogen levels fall precipitously. The skin shows the effects of depleted oestrogen, with reported skin dryness, accelerated worsening of wrinkles and a loss of firmness being key patient and consumer concerns. These concerns correspond with changes in structural and architectural components (such as collagen abundance) as well as decreased sebum production. Interestingly, collagen levels in the skin correlate with oestrogen and supplementation with systemic hormone replacement therapy has been shown to increase collagen synthesis. Oestrogen is important for the normal functioning of many structures in the epidermis and dermis, including the vasculature, hair follicles, sebaceous/apocrine glands, eccrine glands and melanocytes, acting via oestrogen receptors, the beta receptor being more prevalent in the skin. Keratinocytes possess oestrogen receptors which are believed important regulators of differentiation, proliferation and epithelialisation. The changes in epidermal and stratum corneum thickness observed in post-menopausal skin which are partially rescued in HRT cohorts, suggest an important role for oestrogen in epidermal homeostasis. The inventors submit that these mechanisms are behind the visible appearance changes observed in menopausal skin. An impact on skin barrier function can lead to dryness; impact on epidermal renewal processes leads to loss of skin radiance and luminosity as well as rougher skin.

### Cortisol

Applying a hormonal insult may comprise applying cortisol.

Psychological stress is also known to exacerbate various skin diseases including psoriasis, atopic dermatitis and acne. Central to the heightened inflammation are changes in cortisol levels that in turn can impact sebum production in acne and skin barrier function in atopic dermatitis. In studies, final exam and interview stress have been shown to impact barrier function, the latter associated with increased plasma cortisol levels and upregulated inflammation. Skin is a target for cortisol; indeed the skin is also able to produce its own corticosteroids, with corticosteroid (CR) receptors available in the skin to mediate biological effects.

### Melanocyte Stimulating Hormone (MSH)

Applying a hormonal insult may comprise applying MSH. MSH is both locally produced in the skin and is produced by the anterior pituitary. Increased expression in pregnancy is linked to the associated increases in skin pigmentation often seen.

### Pharmaceutical Composition

The compositions of the present invention may be employed together with a pharmaceutical composition, and thereby counteract side effects of the pharmaceutical composition. Typically, the pharmaceutical composition will be administered to the skin (topical application). However, the pharmaceutical composition may be delivered orally.

The pharmaceutical composition may comprise a retinoid, benzoyl peroxide, and/or azelaic acid (nonanedioic acid). The pharmaceutical composition may be described as “prescription strength”.

Retinoids include first generation retinoids (e.g. retinol, retinal, tretinoin (retinoic acid), isotretinoin, and alitretinoin); second generation retinoids (e.g. etretinate and its metabolite acitretin); third generation retinoids (e.g. adapalene, bexarotene, and tazarotene) and fourth generation retinoids (e.g. Trifarotene).

In particular, the pharmaceutical composition may comprise trans-retinoic acid (tretinoin), 13-cis retinoic acid (isotretinoin), and/or adapalene (differin) and tazarotene. The pharmaceutical composition may comprise at least 0.05 wt % retinoids, such as at least 0.05 wt % tretinoin.

The pharmaceutical composition may comprise benzoyl peroxide, such at least 3 wt % or 5 wt % benzoyl peroxide.

The pharmaceutical composition may comprise azelaic acid, such at least 10 wt %, 15 wt % or 20 wt % azelaic acid.

The invention is further described by the following examples and figures in which

FIG. 1 is a schematic hypothetical diagram to demonstrate the effect of a test composition on hormonally impacted skin;

FIG. 2 is a schematic hypothetical diagram to demonstrate how skin sensitivity can be investigated; test compositions are tested their ability to inhibit inflammatory cytokines.

### EXAMPLE FORMULATIONS

The following are examples of compositions according to the invention. They are provided as exemplary compositions only and are not intended to be limiting on the invention.

### Cream Cleanser—Ingredients

| | % w/w |
|---|---|
| Aqua (water) | 76.53 |
| Caprylic/capric triglyceride | 5 |
| Glycerin | 5 |
| Cetearyl alcohol | 2.5 |
| Isononylisononanoate | 2.5 |
| Annona cherimola fruit extract | 2 |
| Glyceryl stearate | 1.2 |
| Butyrospermum parkii (Shea) butter | 1 |
| Niacinamide | 1 |
| Cetearyl alcohol | 1 |
| Phenoxyethanol | 0.5 |
| Polyacrylate crosspolymer-6 | 0.4 |
| Saccharide isomerate | 0.3 |
| Butylene glycol | 0.2 |
| Sodium benzoate | 0.1 |
| Ethylhexylglycerin | 0.1 |
| Bisabolol | 0.1 |
| Antioxidants | 0.5 |
| Tetrasodium EDTA | 0.05 |
| Biphenyl azepanyl methanone | 0.01 |
| Ceramides | 0.01 |

### Cream Cleanser—Method of Manufacture

1. In a main vessel add Aqua (water), Glycerin, Tetrasodium EDTA, to make the water phase.
2. Separately weigh out Butyrospermum parkii (Shea) butter, Isononyl isononanoate, Glyceryl stearate, Cetearyl alcohol, Caprylic/capric triglyceride, Polyacrylate crosspolymer-6 to make the oil phase.
3. Heat both phases to 70-80° C., add the oil phase to the water phase with homogenisation for 5 minutes at 3500 rpm
4. With the emulsion below 35° C., add with stirring: Bisabolol, Biphenyl azepanyl methanone, Saccharide isomerate, Annona cherimola fruit extract, Ceramides, Niacinamide, Sodium benzoate, Phenoxyethanol.

### Gel Cleanser—Ingredients

| | % w/w |
|---|---|
| Aqua (Water) | 72.6 |
| Glycerin | 6 |
| Cocamidopropyl betaine | 5 |
| Lauryl glucoside | 4 |
| Disodium laureth sulfosuccinate | 2.5 |
| Sodium cocoamphoacetate | 2 |
| Coco-glucoside | 1.5 |
| Annona cherimola fruit extract | 1.5 |
| Glyceryl oleate | 1 |
| Niacinamide | 1 |
| PEG-7 Glyceryl stearate | 0.5 |
| Saccharide isomerate | 0.5 |
| Propanediol | 0.5 |
| Phenoxyethanol | 0.4 |
| Panthenol | 0.25 |
| Sodium benzoate | 0.2 |
| Bisabolol | 0.2 |
| Tetrasodium EDTA | 0.1 |
| Sodium hyaluronate | 0.1 |
| Ceramides | 0.1 |
| Ginseng | 0.05 |

### Gel Cleanser—Method of Manufacture

1. In the main vessel add Aqua (water), Glycerin, Panthenol, Tetrasodium EDTA, Sodium Hyaluronate, Phenoxyethanol, Cocamidopropyl betaine, Sodium cocoamphoacetate, Coco-glucoside, Glyceryl oleate, Disodium laureth sulfosuccinate, Lauryl glucoside. Mix until dissolved.
2. Add to the main vessel: Bisabolol, Saccharide isomerate, Biphenyl azepanyl methanone, Annona cherimola fruit extract, Ceramides, Niacinamide, Panax ginseng root extract, Sodium benzoate. Mix until dissolved.
3. Add to the main vessel: PEG-7 Glyceryl stearate. Stir until fully dissolved.

### Rich Moisturiser—Ingredients

| Ingredient | % w/w |
|---|---|
| Aqua (Water) | 66.5 |
| Glycerin | 10 |
| Butyrospermum parkii (Shea) butter | 5 |
| Annona cherimola fruit extract | 3 |
| C12-15 Alkyl benzoate | 2.5 |
| Cetearyl alcohol | 2.5 |
| Dimethicone | 2 |
| Niacinamide | 2 |
| Isononylisononoate | 1.5 |
| Sodium polyacrylate | 1 |
| Propanediol | 1 |
| Saccharide isomerate | 1 |
| Phenoxyethanol | 0.5 |
| Panthenol | 0.5 |
| Tocopherol acetate | 0.2 |
| Butylene glycol | 0.2 |
| Bisabolol | 0.2 |
| Sodium benzoate | 0.1 |
| Sodium hyaluronate | 0.1 |
| Ceramides | 0.1 |
| Tetrasodium EDTA | 0.05 |
| Biphenyl azepanyl methanone | 0.05 |

### Rich Moisturiser—Method of Manufacture

1. In the main vessel add Aqua (water), Glycerin, Tetrasodium EDTA and Sodium Hyaluronate to make the water phase.
2. Weigh out separately: Butyrospermum parkii (Shea) butter, Dimethicone, Tocopheryl acetate, Cetearyl alcohol, Isononyl isononanoate, C12-15 alkyl benzoate, Sodium polyacrylate, Bisabolol. Heat to 70-80° C. with stirring until fully dispearsed.
3. Add the oil phase to the water phase with homogenisation at 3500 rpm for 5 minutes.
4. With the emulsion below 35° C., add with stirring: Phenoxyethanol, Panthenol, Biphenyl azepanyl methanone, Saccharide isomerate, Annona cherimola fruit extract, Ceramides, Niacinamide, Sodium benzoate.

### Investigation

The inventors submit that inflammatory mechanisms operating in the skin underly different manifestations of sensitivity. Activation of the Hypothalamic Pituitary Axis (HPA) with psychological stress results in the release of cortisol and related hormones that affect gene expression in skin cells by, for example, binding to transcription factors such as Activating Protein-1 (AP-1) and NFK-beta, downstream factors essential in the induction of inflammation. Epidermal skin cells respond to cortisol by producing Interleukin-6 and other pro-inflammatory cytokines. Other cells such as sebocytes which produce sebum also respond to stress hormones by producing pro-inflammatory mediators. Similarly, other hormones such as the oestrogens through epidermal receptors play a role in the regulation of inflammation. Reduced levels or deprivation in post-menopausal skin results in an increased low level inflammatory state that is likely to link to greater perceptions of skin sensitivity. Transient receptor potential vanilloid (TRYPV-1) “Capsaicin receptor” ion channel is found on sensory nerve endings and other non-neuronal cells such as keratinocytes and sebocytes where it regulates calcium influx into cells. Its activation especially via histamine, is associated with itch. Again, hormones such as oestrogen are known to exert some of their roles via TRYPV-1 so it is not surprising that hormonal fluctuations can result in skin sensitivity such as itch through this mechanism. Skin responsiveness to capsaicin, a natural activator of TRPV1, has been shown to correlate with sensitive skin.

### Cell Culture Model

A skin cell culture model was developed to simulate the impact of hormones and hormone deprivation/fluctuation on skin cell behaviour. Skin cells are cultured in media that is conditioned with hormones (such as oestrogen and oestradiol derivates, cortisol and corticosteroid derivatives). Biomarkers of cell health and performance are measured including but not limited to proliferation rate, oxidative stress, inflammatory biomarkers, and cell viability.

Test compositions (comprising suspected active ingredients) are added into the media and a change in cell behaviour measured. In particular active ingredients can be evaluated for their ability to mimic the effect of hormones and improve the skin cell resilience against hormonal fluctuations.

For example, test compositions could be assessed for the ability to inhibit IL-6 or TRYPV-1.

### IL-6 Inhibition Assay

Primary human keratinocytes, fibroblasts, or immortalised keratinocytes (HaCATs) are seeded in a 96 well plate at a density of 5,000 cells per well (according to QPRO/148 method for determining cell numbers) in cell growth media with supplements and allowed to incubate for 24 hours at 37° C. After incubation, the cell growth media is replaced with 100 μl of Phosphate Buffered Saline (PBS) without calcium and magnesium and containing the relevant concentration of active required. Controls include untreated irradiated and non-irradiated samples which require 100 μl pure PBS without calcium and magnesium or containing a maximum of 0, 1% Dimethyl sulfoxide (DMSO) if required to dissolve actives. Salicylic acid at 0.01% is used as the positive control. Cells are incubated with the active ingredients for 30 min at 37° C. before irradiating the cells with a UV dose of 61,500 J/m². After irradiation, the PBS and active ingredients are replaced with 100 μl of pre-warmed (37° C.) media without supplements and incubated for 24 hrs at 37° C. Media supernatant containing the IL-6 expressed from the cells is then collected and transferred to a fresh 96 well plate and stored at −20° C. until the ELISA is performed. The IL-6 ELISA should be performed according to the manufacturer's protocol provided with the kit.

### TRYPV-1 Inhibition Assay

After 24 h, the cell culture medium was replaced by DMEM containing 2 μM calcium fluorescent probe Fluo-3 and 0.04% Pluronic F127 (Beijing FanBo Biochemicals Co., Ltd) and incubated for 30 min. Various concentrations of phenoxyethanol and capsaicin were added, with 3 mM of EGTA (ethylene glycol tetraacetic acid; Sigma, U.S.A.) as negative control and 0.01% Triton X-100 (Sigma, U.S.A.) as positive control 24. To confirm the role of TRPV1, 7.8 μg mL-1 ID1609 (trans-tert-butyl cyclohexanol (TTBC) in pentylene glycol, marketed as SymSitive®1609) with 250 μg mL-1 phenoxy ethanol or with 40 nM capsaicin (Symrise, Germany) was also added. Twenty minutes later, the cells were observed under fluorescence microscope. ImageJ®software was used to analyse the fluorescence intensity of each image. The calcium influx was calculated according to the formula [Ca++]i=Kd (F−Fmin)/(Fmax−F), with Kd value of Fluo-3 as 400 nM, F as mean fluorescence intensity of different culturing conditions, Fmin as the fluorescence intensity of the negative controls and Fmax as the fluorescence intensity of the positive controls. The experiment was repeated at least three times for each culturing condition.

Hypothetical results are illustrated in FIG. 2 and the table below.

| | % IL-6 | % TRYPV-1 |
|---|---|---|
| Active ingredient/complex | inhibition | Inhibition |
| Bisabolol | 50 | 3 |
| Niacinamide | 10 | 7 |
| Biphenyl azepanyl methanone (BAM) | 8 | 5 |
| Cherimoya | 15 | 42 |
| Bisabolol + Niacinamide + Biphenyl | 76 | 39 |
| azepanyl methanone | | |
| Bisabolol + Niacinamide + Biphenyl | 84 | 61 |
| azepanyl methanone + Cherimoya | | |

1. A composition comprising
(i) a monocyclic sesquiterpene alcohol;
(ii) vitamin B or a derivative thereof; and
(iii) an amide of formula (I) 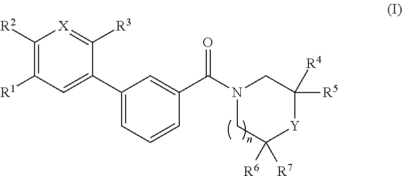
wherein X is CH or N,
Y is CHR⁸ or O, n is 0, 1 or 2,
R¹, R² and R³ are independently of each other selected from the group consisting of H, OH, a halogen atom, a carbamoyl group and C₁-C₆ alkyl group, and
R⁴, R⁵, R⁶, R⁷ and R⁸ are independently of each other H or a C₁-C₆ alkyl group.

2. The composition of claim 1, wherein
(i) the monocyclic sesquiterpene alcohol is present in the composition in an amount by weight of from 0.001% to 2%; and/or
(ii) the vitamin B or a derivative thereof is present in the composition in an amount by weight of from 0.01% to 5%; and/or
(iii) the amide of formula (I) is present in the composition in an amount by weight of from 0.001% to 1%.

3. The composition of claim 1, additionally comprising an Annona extract.

4. The composition of claim 3, wherein the Annona extract is present in the composition in an amount by weight of from 0.1% to 5%.

5. The composition of claim 1, wherein the monocyclic sesquiterpene alcohol comprises bisabolol.

6. The composition of claim 1, wherein the vitamin B or derivative thereof is a vitamin B3 compound having the formula: 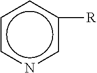
wherein R is —CONH₂ (niacinamide), —COOH (nicotinic acid) or CH₂OH (nicotinyl alcohol); or an ester or salt thereof.

7. The composition of claim 1, wherein the amide of formula (I) is chosen from one or more in the group of (4-methylpiperidin-1-yl) (3-(6-methylpyridin-3-yl)phenyl) methanone, (4′-hydroxy-[1,1′-biphenyl]-3-yl) (4-methylpiperidin-1-yl) methanone, (4′-fluoro-[1,1-biphenyl]-3-yl) (4-methylpiperidin-1-yl) methanone, (3′-fluoro-4′-methyl-[1,1-biphenyl]-3-yl) (4-methylpiperidin-1-yl) methanone, (2′-chloro-[1,1′-biphenyl]-3-yl) (4-methylpiperidin-1-yl) methanone, (4′-methyl-[1,1′-biphenyl]-3-yl) (4-methylpiperidin-1-yl) methanone, (4′-chloro-[1,1 biphenyl]-3-yl) (4-methylpiperidin-1-yl) methanone, (3,3-dimethylpiperidin-1-yl) (3′-fluoro-4′-methyl-[1,1′-biphenyl]-3-yl) methanone, 3′-(4-methylpiperidine-1-carbonyl)-[1,1′-biphenyl]-4-carboxamide, (3′-hydroxy-[1,1′-biphenyl]-3-yl) (4-methylpiperidin-1-yl) methanone, 3′-(4-methylpiperidine-1-carbonyl)-[1,1-biphenyl]-3-carboxamide, (2,2-dimethylmorpholino) (3′-fluoro-4′-methyl-[1,1′-biphenyl]-3-yl) methanone, (2,6-dimethylmorpholino) (3′-fluoro-4′-methyl-[1,1-biphenyl]-3-yl) methanone, (2,6-dimethylmorpholino) (4′-methyl-[1,1 biphenyl]-3-yl) methanone, azepan-1-yl (3′-fluoro-4′-methyl-[1,1′-biphenyl]-3-yl) methanone, azepan-1-yl (4′-chloro-[1,1′-biphenyl]-3-yl) methanone, azepan-1-yl (4′-methyl-[1,1′-biphenyl]-3-yl) methanone, azepan-1-yl (4′-hydroxy-[1,1′-biphenyl]-3-yl) methanone, azepan-1-yl (3-(6-methylpyridin-3-yl)phenyl) methanone, [1,1′-biphenyl]-3-yl (azepan-1-yl) methanone, and azepan-1-yl (3′,4′-dimethyl-[1,1′-biphenyl]-3-yl) methanone.

8. The composition of claim 1 wherein
(i) the monocyclic sesquiterpene alcohol comprises α-bisabolol;
(ii) the vitamin B or a derivative thereof comprises niacinamide; and/or
(ii) the amide of formula (I) comprises biphenyl azepanyl methanone.

9. The composition of claim 1, wherein the composition comprises Annona cherimola fruit extract.

10. The composition of claim 1, wherein α-bisabolol is present in amount by weight of from 0.05 to 0.15, niacinamide, biphenyl azepanyl methanone is present in amount by weight of from 0.1 to 5.0, and Annona cherimola fruit extract is present in amount by weight of from 1.0 to 5.0.

11. The composition of claim 1, additionally comprising a ginseng extract; at least one ceramide; saccharide isomerate; hyaluronic acid or a salt thereof; and/or a Trifolium extract.

12. An in vitro method for assessing the effect of a test composition on hormonally impacted skin, the method comprising:
a) applying a hormonal insult to skin cells in vitro to yield hormonally impacted skin cells;
b) applying a test composition to the hormonally impacted skin cells; and
c) measuring a change in a property of the hormonally impacted cells.

13. A cosmetic method for preventing and/or treating skin sensitivity in a person in need thereof, the method comprising topically administering a composition as defined in claim 1.

14. A kit comprising the composition of claim 1 and a pharmaceutical composition.

15. The kit of claim 14 for use in a method for the treatment of a skin condition, wherein the composition is applied to the skin of a person in need thereof before the pharmaceutical composition; simultaneously with the pharmaceutical composition; or after the pharmaceutical composition.
